(19) United States
(12) Patent Application Publication
(10) Pub. No.: US 2021/0353298 A1
(43) Pub. Date: Nov. 18, 2021

(54) Flow rate control device for variable artery occlusion

(71) Applicant: Government of the United States as represented by the Secretary of the Air Force, Wright-Patterson AFB, OH (US)

(72) Inventors: Timothy K Williams, Winston-Salem, NC (US); Lucas Paul Neff, Winston-Salem, NC (US)

(73) Assignee: Government of the United States as represented by the Secretary of the Air Force, Wright-Patterson AFB, OH (US)

(21) Appl. No.: 17/318,549

(22) Filed: May 12, 2021

Related U.S. Application Data
(63) Continuation of application No. 16/305,991, filed on Nov. 30, 2018, now abandoned.
(60) Provisional application No. 62/345,825, filed on Jun. 5, 2016.
(60) Provisional application No. 62/365,155, filed on Jul. 21, 2016.

(51) Int. Cl. A61B 17/12; A61M 29/02; A61M 25/10; A61M 25/00

(52) U.S. Cl. CPC A61B 17/12036; A61B 17/1204; A61B 17/12109; A61B 17/12136; A61B 2090/061; A61M 25/10; A61M 25/0097; A61B 2017/1205; A61M 2025/1095; A61M 29/02

(57) ABSTRACT

An endovascular occlusion device. The endovascular occlusion device (300) has a balloon (306) and a catheter (304). The catheter (304) has a distal end (308), a proximal end, and a lumen (318) extending therebetween. The balloon (306) is positioned proximate to the distal end (308) of the catheter (304) and has a deflated state and an inflated state. The catheter (304) further includes a plurality of ports (314) proximate to a proximal end of the balloon (306). Each port (314) extends through a wall of the catheter (304) such that surface (316) of the catheter (304) is in fluid communication with the lumen (318) of the catheter (304). A flow restrictor (324) is positioned within, and is in sliding relation with, the lumen (318) of the catheter (304). Movement of the flow restrictor (324) is configured to close one or more ports (314) of the plurality so as to limit blood flow through the lumen (318) of the catheter (304).

This application is a continuation of U.S. application Ser. No. 16/305,991, filed 30 Nov. 2018, which was the U.S. National Stage Application of International Application No. PCT/US17/36023 filed Jun. 5, 2017, which claimed the benefit of and priority to prior filed co-pending Provisional Application Ser. No. 62/345,825, filed Jun. 5, 2016, and prior filed co-pending Provisional Application Ser. No. 62/365,155, filed Jul. 21, 2016. The disclosure of each of these applications is expressly incorporated herein by reference, each in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to surgical devices and, more particularly, to surgical devices suitable for arterial occlusion.

### BACKGROUND OF THE INVENTION

Slowing a rate of blood loss for a severely injured patient is critical in saving that patient's life. Conventionally, slowing the rate of blood loss has been accomplished by limiting (or even stopping) the flow of blood through any major blood vessel leading to the site of blood loss. For medics in a battlefield or a first responder setting, slowing the loss of blood of a patient having significant lower body injury has been achieve by aortic occlusion—using a large aortic clamp that is inserted into the chest cavity via a large incision between the ribs. The goal of the aortic clamping procedure is to keep the patient's remaining blood circulating between the heart, lungs, and brain until bleeding below the aortic clamp is controlled and systemic circulation restored. By clamping the aorta, systemic circulation is excluded, causing an ischemia. Thus, the highly invasive maneuver of aortic clamping is often a “last ditch” effort, used only for the most injured patient having lost vital signs and are considered, practically, clinically dead.

Conventional balloon catheters used in endovascular surgery have recently been repurposed to fully occlude the aorta by inflation of the balloon and as an alternative to aortic clamping. This procedure, referred to as Resuscitative Endovascular Balloon Occlusion of the Aorta (“REBOA”), has the potential to achieve effective aortic occlusion with a lower rate of morbidity. Thus it is believed that REBOA may be used earlier in the clinical course of the bleeding patient as compared to the conventional aortic clamp procedure.

Because blood flow is restricted from tissues below the aortic occlusion, tissues of that region start to die due to lack of blood flow. Therefore, as soon as is feasible after successful use of aortic occlusion (whether by clamp or balloon) and loss of blood is controlled, the patient is “weaned” from full occlusion. Unfortunately, current, FDA-approved balloon catheters suitable for REBOA are capable of achieving only complete occlusion or no occlusion. Further complicating matters is that as the REBOA balloon is deflated to initiate flow, hemodynamic collapse is a possibility. Moreover, if patient size (height, weight, aortic diameter) requires the use of multiple REBOE balloons, then the risk of hemodynamic collapse occurs with deflation of each balloon.

Accordingly, there remains a need for medical devices configured to effectively and efficiently control endovascular occlusion of arteries in both the trauma setting and the clinical setting.

### SUMMARY OF THE INVENTION

The present invention overcomes the foregoing problems and other shortcomings, drawbacks, and challenges of conventional endovascular occlusion devices. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention, an endovascular occlusion device has a balloon and a catheter. The catheter has a distal end, a proximal end, and a lumen extending therebetween. The balloon is positioned proximate to the distal end of the catheter and has a deflated state and an inflated state. The catheter further includes a plurality of ports proximate to a proximal end of the balloon. Each port extends through a wall of the catheter such that surface of the catheter is in fluid communication with the lumen of the catheter. A flow restrictor is positioned within, and is in sliding relation with, the lumen of the catheter. Movement of the flow restrictor is configured to close one or more ports of the plurality so as to limit blood flow through the lumen of the catheter.

In other embodiments of the present invention, an endovascular occlusion device includes a first balloon and a second balloon. Each of the first and second balloons has a distal end, a proximal end, and a lumen extending therebetween. The first and second balloons each also have a deflated state and an inflated state. When the second balloon is in the inflated state, blood flow through the lumen of the first balloon is restricted. When the second balloon is in the deflated state, blood may flow through the lumen of the first balloon.

Still other embodiments of the present invention include an endovascular occlusion device having a first balloon, a second balloon, and an inflatable plug. The first balloon has a distal end, a proximal end, and a lumen extending therebetween; the first balloon has a deflated state and an inflated state. The second balloon has a distal end, a proximal end, and a lumen extending therebetween; the second balloon is coaxial with the first balloon and has a deflated state and an inflated state. The inflatable plug has a distal end and a proximal end; the inflatable plug is coaxial with the first and second balloons and has a deflated state and an inflated state. When the inflatable plug is in the inflated state, the inflatable plug forms a seal with the second balloon.

Yet other embodiments of the present invention include an endovascular occlusion device having a first balloon and a second balloon. The first balloon has a distal end and a proximal end; the first balloon also has a deflated state and an inflated state. A channel extends between the distal and proximal ends of the first balloon and radially inwardly from an outer surface of the first balloon. The channel has a first side and a second side. The second balloon has a distal end and a proximal end and is in juxtaposition with the channel of the first balloon. The second balloon has a deflated state and an inflated state. When the second balloon is in the inflated state, the second balloon moves the first and second sides of the channel in opposing directions so as to open the channel.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a diagrammatic view of an exemplary method of accessing the abdominal aorta for performing vascular occlusion, shown in partial cross-section.

FIG. 2 is a side elevational view of an endovascular occlusion device according to an embodiment of the present invention.

FIGS. 3-6 are perspective views of a balloon portion of the endovascular occlusion device illustrated in FIG. 2.

FIGS. 3A-6A are cross-sectional view of the balloon portion of the endovascular occlusion device taken along respective A-A lines of FIGS. 3-6.

FIGS. 7A-7D are sequential diagrammatic views of a method occluding an artery with the balloon portion illustrated in FIGS. 3-6A according to one embodiment of the present invention.

FIG. 8 is a side elevational view of an occluding portion of an endovascular occlusion device according to another embodiment of the present invention.

FIG. 9 is a disassembled, side elevational view of the occluding portion of FIG. 11.

FIG. 10 is a perspective view of the occluding portion of FIG. 8.

FIGS. 11A and 11B are sequential diagrammatic view of a method of occluding an artery with the occluding portion of FIG. 8 according to an embodiment of the present invention.

FIGS. 12A-12C are sequential diagrammatic views of a method of occluding an artery with the occluding portion of FIG. 8 according to another embodiment of the present invention.

FIGS. 13A and 13B are sequential diagrammatic views of a method of occluding an artery with the occluding portion of FIG. 8 according to still another embodiment of the present invention.

FIG. 14 is a perspective view of an occluding portion of an endovascular occlusion device according to an embodiment of the present invention.

FIG. 15 is a disassembled, top perspective view of the occluding portion of FIG. 14.

FIG. 16 is an assembled, top perspective view of the occluding portion of FIG. 14 with the occluding portion configured to permit blood flow therethrough.

FIG. 17 is a top view of the occluding portion as illustrated in FIG. 16.

FIG. 18 is an assembled, top perspective view of the occluding portion of FIG. 14 with the occluding portion configured to prevent blood flow therethrough.

FIGS. 19A-19C are sequential diagrammatic views of a method of occluding an artery with the occluding portion of FIG. 14 according to one embodiment of the present invention.

FIGS. 20A, 21A, 22A, and 23A are perspective views of an occluding portion of an endovascular occlusion device according to another embodiment of the present invention.

FIGS. 20B, 21B, 22B, and 23B are longitudinal, cross-sectional view of the occluding portion of FIGS. 20A, 21A, 22A, and 23A, respectively.

FIGS. 20C, 21C, 22C, and 23C are transverse, cross-sectional view of the occluding portion of FIGS. 20A, 21A, 22A, and 23A, respectively.

FIGS. 24A-24D are sequential diagrammatic views of a method of occluding an artery with the occluding portion of FIG. 20A according to one embodiment of the present invention.

FIG. 25 is a disassembled, perspective view of a control handle according to an embodiment of the present invention, shown in partial cross-section.

FIG. 26 is an assembled, perspective view of the control handle of FIG. 25, shown in partial cross-section.

FIG. 27 is a top view of the control handle of FIG. 26, shown in partial cross-section.

FIG. 28 is a side elevational view of an occluding portion of an endovascular occlusion device according to still another embodiment of the present invention.

FIG. 29 is a disassembled view of the occluding portion shown in FIG. 28.

FIG. 30 is a transverse, cross-sectional view of the flow port catheter taken along the line 30-30 of FIG. 28.

FIG. 31 is a longitudinal, cross-sectional view of the flow port catheter taken along the line 31-31 of FIG. 28.

FIGS. 32 and 33 are sequential diagrammatic views of a method of using the occluding portion of FIG. 28 according to one embodiment of the present invention.

FIGS. 32A and 33A are cross-sectional views of FIGS. 32 and 33, respectively, and in a manner similar to FIG. 31.

FIGS. 34 and 35 are sequential diagrammatic views of a method of using the occluding portion of FIG. 28 according to another embodiment of the present invention.

FIGS. 34A and 35A are cross-sectional views of FIGS. 34 and 35, respectively, and in a manner similar to FIG. 31.

FIGS. 36A and 36B are perspective views illustrating an appliance configured to clear ports of occluding portion illustrated in FIG. 28.

FIG. 37 is a side elevational view of a handle suitable for use with the occluding portion illustrated in FIG. 28.

FIGS. 38A and 38B are side elevational views illustrating a method of using the handle of FIG. 37.

FIG. 39 is an enlargement of a portion within enclosure 39 of FIG. 38B.

FIGS. 40-43 are graphical representations of experiment data obtained while modeling a pig aorta and using an endovascular occlusion device according to an embodiment of the present invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

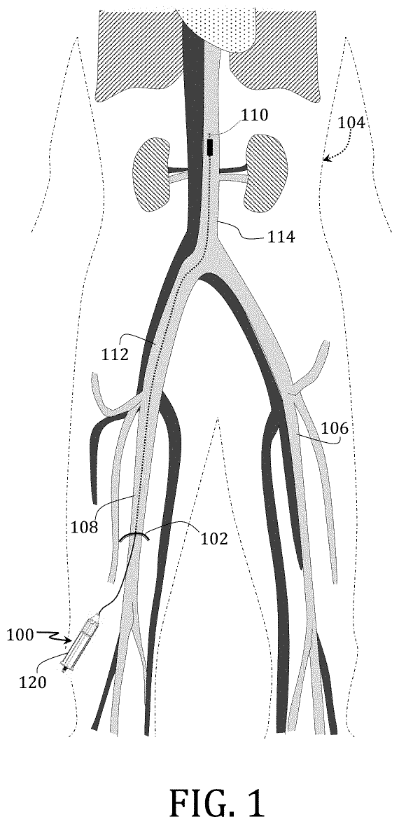

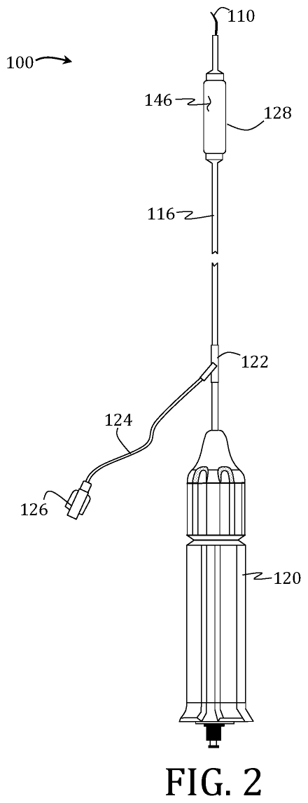

Referring now to the figures, and in particular to FIGS. 1 and 2, a method of using an endovascular occlusion device 100 according to a first embodiment of shown. While the illustrative embodiment applies to aortic occlusion, the surgeon having ordinary skill in the art and the benefit of the disclosure herein will readily understand how to implement similar methods and devices to other endovascular occlusions.

The method begins with the surgeon making a primary incision site 102 in the patient 104 that is substantially near a superficial vein. A suitable superficial vein for the primary incision site 102 can include a peripheral vein, on either of the right or left sides of the patient 104, such as the left or right femoral artery 106, 108, or others known by one skilled in the art. Similar veins or locations on the left side of the body could also be used.

The surgeon may then direct a guidewire 110 (for example, a 0.025 in guidewire) into the primary incision site 102, within the right femoral artery 108, superiorly through the common iliac artery 112, and up the abdominal aorta 114 to a desired location and site for occlusion (hereafter, the “occlusion site”). With the guidewire 110 suitably positioned, the endovascular occlusion device 100 may be back-loaded over the guidewire 110 and advanced to the location of occlusion.

The endovascular occlusion device 100, as shown in FIG. 2, includes a catheter 116 having a distal balloon portion 118 and a proximally positioned handle 120. As shown, the handle 120 is a manual flow control handle, described in greater detail below. Distal to the handle 120, a y-joint 122 is coupled to an inflation line 124 by way of a luer lock 126 to the catheter 116 for inflating and collapsing the balloon portion 118.

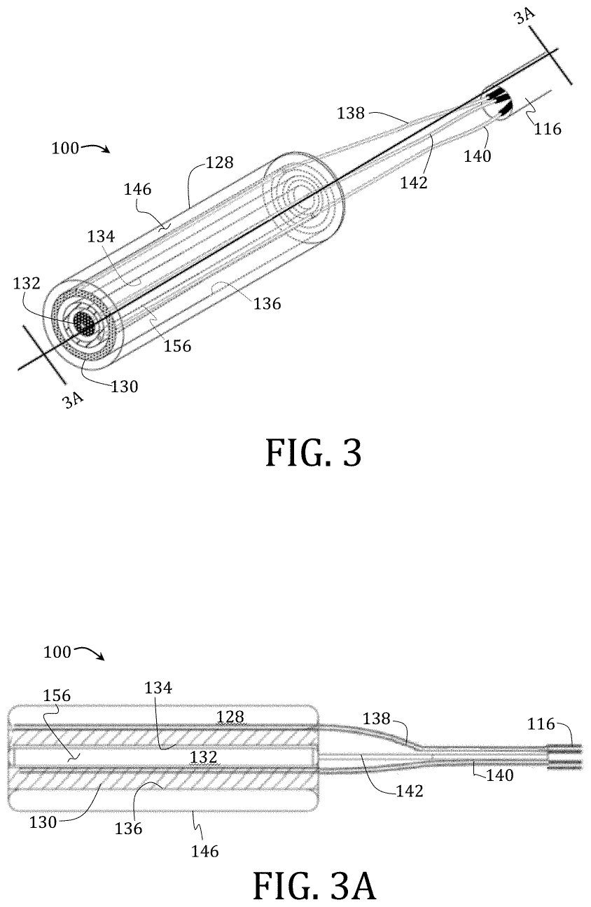

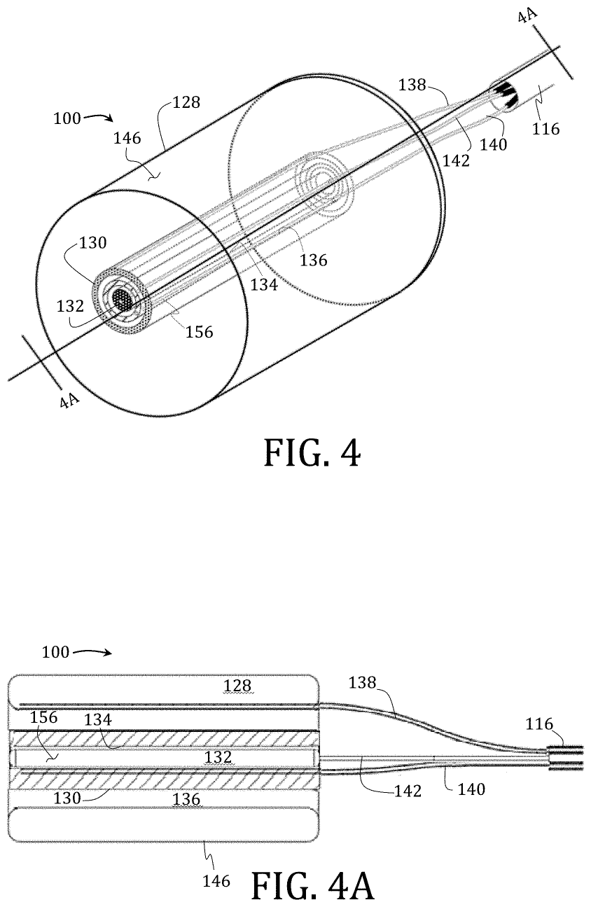

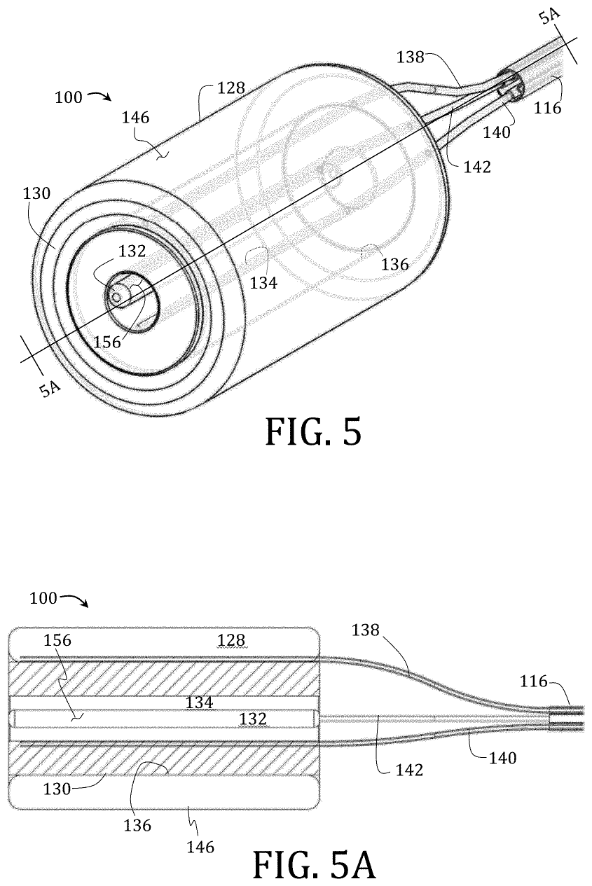

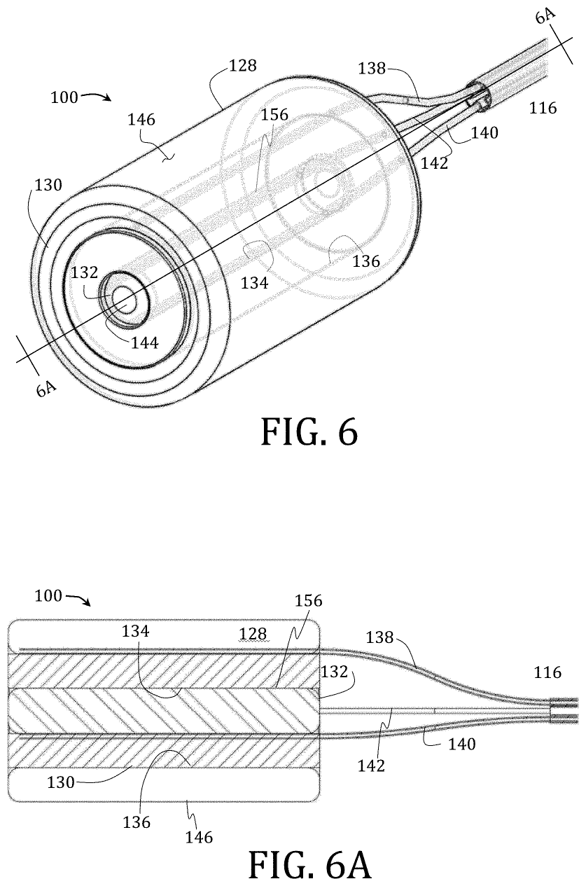

As shown with greater detail in FIGS. 3-6A, the balloon portion 118 of the endovascular occlusion device 100 includes first, second, and third coextensive (and in some embodiments, collinear, coaxial, or both) balloons 128, 130, 132 arranged such that the third catheter balloon 132 resides within a lumen 134 of the second catheter balloon 130, which in turn resides within a lumen 136 of the first catheter balloon 128. Each of the balloons 128, 130, 132 includes a shaft 138, 140, 142 extending proximally therefrom and that is in fluid communication with the inflation line 124 (FIG. 2). The third balloon also includes a lumen 144 that is configured to receive and move in sliding relation to the guidewire 110 (FIG. 1).

The first balloon 128 may be constructed of a compliant or noncompliant material, such as Nylon-11, Nylon-12, polyurethane, polybutylene terephthalate (“PBT”), PEBAX (a brand of thermoplastic elastomer), or polyethylene terephthalate (“PET”), such that when the first balloon 128 is fully inflated an outer surface 146 of the first balloon 128 contacts an inner wall 148 of the artery to be occluded (illustrated in FIG. abdominal aorta 114 in FIG. 1). The first balloon 128 is further configured to expand to outer diameters ranging from 15 mm to 24 mm to accommodate various sizes of vasculature of humans (or sized according to the animal upon which surgery is performed).

The second balloon 130 may be constructed of a compliant material, such as those provided above with respect to the first balloon 128, such that when the second balloon 130 is fully inflated an outer surface 150 of the second balloon 130 contacts the lumen 136 of the first balloon 128.

The third balloon 132 may be constructed of a compliant material, such as those provided above with respect to the first balloon 128, such that when the third balloon 132 is fully inflated an outer surface 152 of the third balloon 132 contacts the lumen 134 of the second balloon 130.

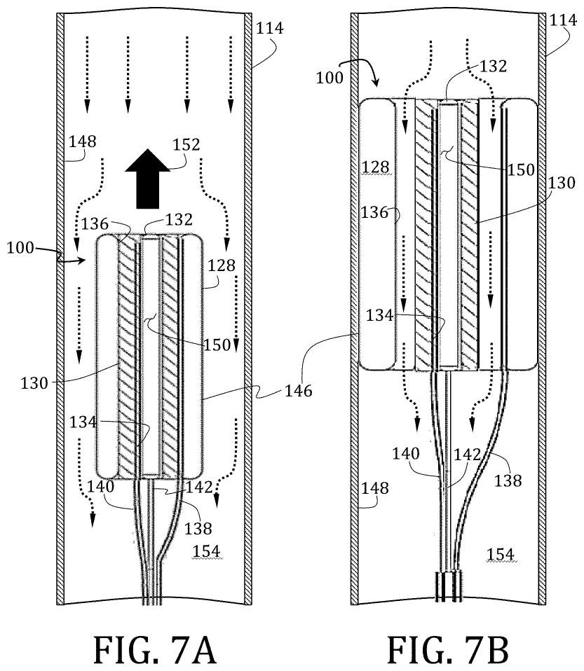

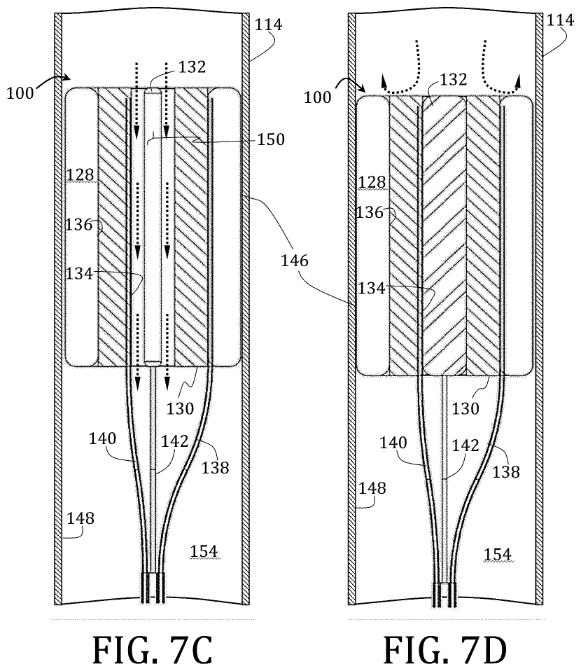

In use, and with reference now to FIGS. 7A-7D, the balloon portion 118 of the occlusion device 100 is advanced in the direction of arrow 152 such that it is suitably positioned within the artery for which occlusion is desired (again, here illustrated as the abdominal aorta 114). Blood flow, as illustrated by dashed arrows, opposes the advancing direction arrow 152. Once in place (FIG. 7B), the first balloon 128 of the balloon portion 118 may be inflated with a fluid, which may be saline with or without a contrast agent to facilitate localization via conventional medical imaging procedures. Blood flow, while somewhat diminished, continues by way of the lumen 136 of the first balloon 128 and around the outer surface 150 of the second balloon 130. Inflation of the first balloon 128, while limiting blood flow, provides the additional benefit of securing the balloon portion 118 within a lumen 154 of the abdominal aorta 114.

With specific reference to FIG. 7C, the second balloon 130 of the balloon portion 118 of the occlusion device 100 may then be inflated in a manner similar to that which was provided above with respect to inflating the first balloon 128. Again, blood flow is further diminished as the remaining path for flow is by way of the lumen 134 of the second balloon 130 and around an outer surface 156 of the third balloon 132.

Finally, in FIG. 7D, the third balloon 132 of the balloon portion 118 of the occlusion device 100 may be inflated (again, in a manner similar to that described above). Inflation of the third balloon 132 reduces fluid flow space within the lumen 134 of the second balloon 130 until full occlusion is achieved, as specifically illustrated.

While not specifically illustrate, deflation and removal of the occlusion device 100 may occur in a manner that is generally the reverse of the illustrative inflation method.

Provided the three balloons 128, 130, 132 of the balloon portion 118 of the occlusion device 100, flow rate of blood along the vessel to be occluded may be controlled with particularity. For example, flow may range from full occlusion, 150 mL/min, 300 mL/min, 500 mL/min, to full flow depending on a degree of inflation of the second and third balloons 130, 132. Such finer control and management of blood flow overcomes several of the deficiencies of conventional devices that fail to offer such functionality.

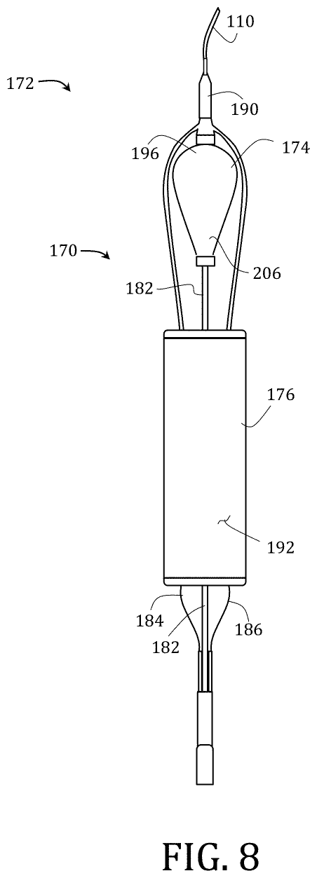

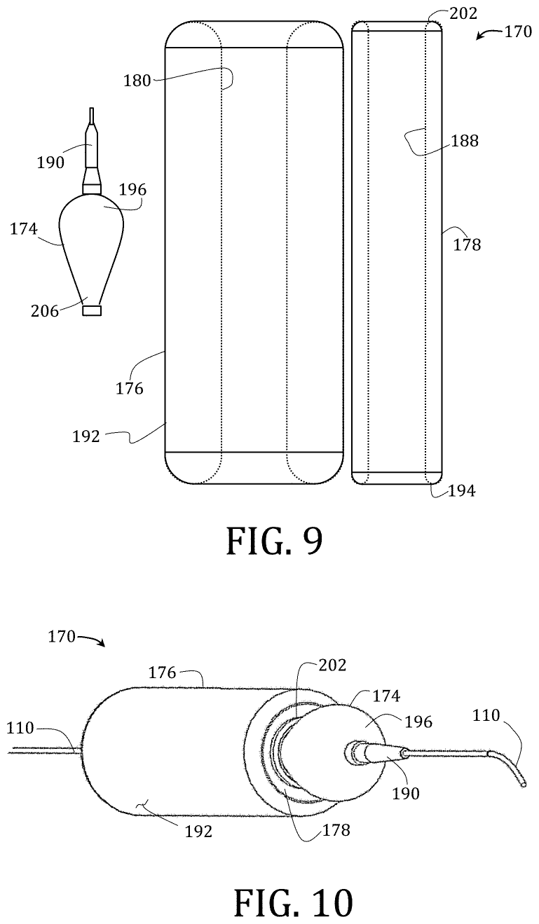

Turning now to FIGS. 8-10 an occluding portion 170 of an endovascular occlusion device 172 suitable for use in both anterograde and retrograde blood flow procedures is described with greater detail. The occluding portion 170 includes an inflatable plug 174, a first balloon 176, and a second balloon 178, wherein the second balloon 178 is coaxial with, and resides within a lumen 180 of, the first balloon 176. Each of the inflatable plug 174 and second balloon 178 may be constructed from non-compliant materials and further includes an inflation catheter 182, 184 extending proximally therefrom. The first balloon 176 may be constructed from a compliant material and also includes an inflation catheter 186 extending proximally therefrom. Compliant and non-compliant materials may include those described in detail above or any other suitable material known by those of ordinary skill in the art having the benefit of the disclosure made herein.

The first and second balloons 176, 178, although not explicitly illustrated here, may be coupled together such that the second balloon 178 is secured within the lumen 180 of the first balloon 176 and such that the first and second balloons 176, 178 move in concert.

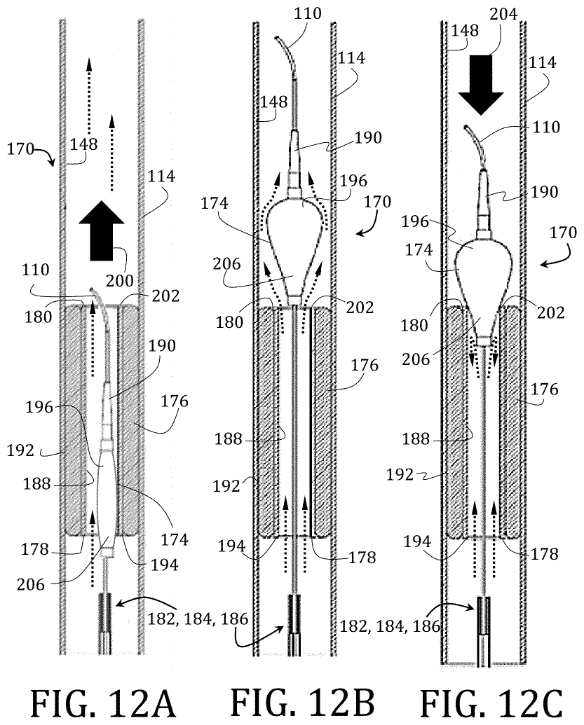

The inflatable plug 174 is configure to deflate (shown in FIG. 12A) to a size suitable to move within and with respect to a lumen 188 of the second balloon 178. Moreover, as provided in the illustrative embodiment, the inflatable plug 176 may further include an obturator 190 (otherwise known by those skilled in the art as an introducer or cone) configured to dilate an opening within a tissue such that medical device (here, the inflatable plug 174) may then pass through such tissue. However, it would be understood by the skilled artisan that the obturator 190 need not be included with the inflatable plug but, rather, may be a commercially-available, standalone device.

According to some embodiments of the present invention, the inflatable plug 174 is physically separated from the first and second balloons 176, 178 such that the inflatable plug 174 and be advanced to the occlusion site sequentially before or sequentially after advancing the first and second balloon 176, 178 to the occlusion site. Alternatively, according to other embodiments of the present invention, the inflatable plug 174 with the first and second balloons 176, 178, forming a conjoined unit that is advanced to the occlusion site as a singular device.

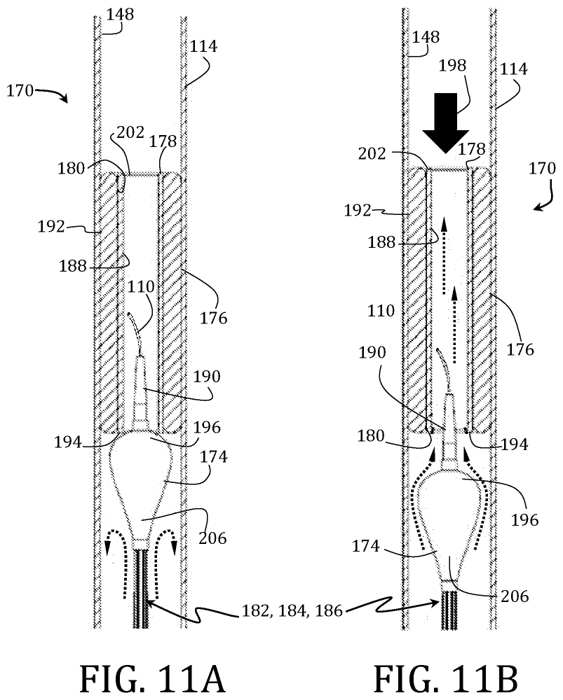

Turning now to FIGS. 11A and 11B, with reference to FIG. 1, a method of using the endovascular occlusion device 172 of FIG. 8 according to an embodiment of the present invention is shown. At start, and as described above with reference to FIG. 1, the guidewire 110 may be inserted into a primary incision site 102 and navigated through the vasculature to an occlusion site, which is illustrated in greater detail in FIGS. 11A and 11B.

With the guidewire 110 in place, the first and second balloons 176, 178 may be advanced over the guidewire 110 to the occlusion site and inflated such that an outer surface 192 of the first balloon 176 contacts the inner wall 148 of the abdominal aorta 114, thereby securing the first and second balloons 176, 178 within the lumen 154 of the abdominal aorta 114.

While maintaining a position of the first and second balloons 176, 178, the inflatable plug 174 may be advanced over the guidewire 110 to the occlusion site but proximal to the inflated first and second balloon 176, 178. The inflatable plug 176 may then be inflated (as shown in FIG. 11A) and advanced to a proximal edge 194 of the second balloon 178. Because the inflatable plug 174 and the second balloon 178 are constructed of non-compliant materials, contact between a distal surface 196 of the inflatable plug 174 and the proximal edge 194 of the second balloon 178 is configured to form a seal against blood flow (illustrated again, here, as dashed lines).

In FIG. 11B, when necessary or desired, the inflatable plug 174 may be retracted slightly (in a direction indicated by arrow 198) such that the distal surface 196 of the inflatable plug 174 is spaced a distance away from the proximal edge 194 of the second balloon 178, thereby releasing the seal of FIG. 11A and permitting blood to flow through the lumen 188 of the second balloon 178 and distally therefrom.

FIGS. 12A-12C illustrate another manner of using the endovascular occlusion device 172 of FIG. 8 according to another embodiment of the present invention. Again, at start, the guidewire 110 may be inserted into a primary incision site 102 and navigated through the vasculature to an occlusion site. With the guidewire 110 in place, the first and second balloons 176, 178 may be advanced over the guidewire 110 to the occlusion site and inflated such that the outer surface 192 of the first balloon 176 contacts the inner wall 148 of the abdominal aorta 114, thereby securing the first and second balloons 176, 178 within the lumen 154 of the abdominal aorta 114.

While maintaining this position of the first and second balloons 176, 178, the inflatable plug 174 may be advanced over the guidewire 110 to the occlusion site and through the lumen 188 of the second balloon 178, as represented by a direction of an arrow 200 in FIG. 12A. Once the inflatable plug 174 clears a distal end 202 of the second balloon 178, the inflatable plug 174 may be inflated (FIG. 12B). Retracting the inflatable plug 174 (as represented by a direction of an arrow 204 in FIG. 12C) places the distal end 202 of the second balloon 178 in contact with a proximal surface 206 of the inflatable plug 174, thereby forming a seal against blood flow (illustrated again, here, as dashed lines). Releasing the seal may accomplished by advancing the inflatable plug 174 distally with respect to the first and second balloons 176, 178 or deflating the inflatable plug 174.

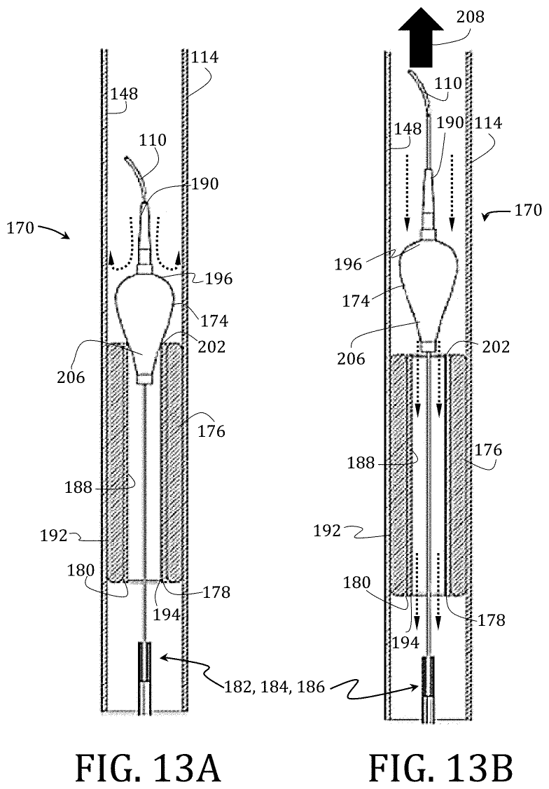

FIGS. 13A and 13B illustrate a method of using the endovascular occlusion device 172 of FIG. 8 according to still yet another embodiment of the present invention and in which a direction of blood flow (illustrated again dashed lined arrows) is in a direction that opposes blood flow in FIGS. 11A-12C. It should be noted that the method illustrated in FIGS. 13A and 13B (and indeed, also the method illustrated in FIGS. 12A-12C), the inflatable plug 174 and the first and second balloons 176, 178 may be advanced, as a unit, to the occlusion site as opposed to the two step method of FIGS. 11A-11B.

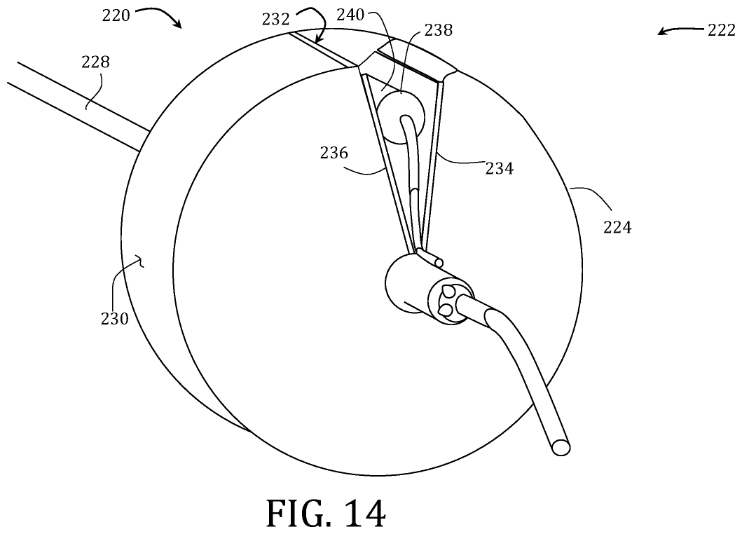

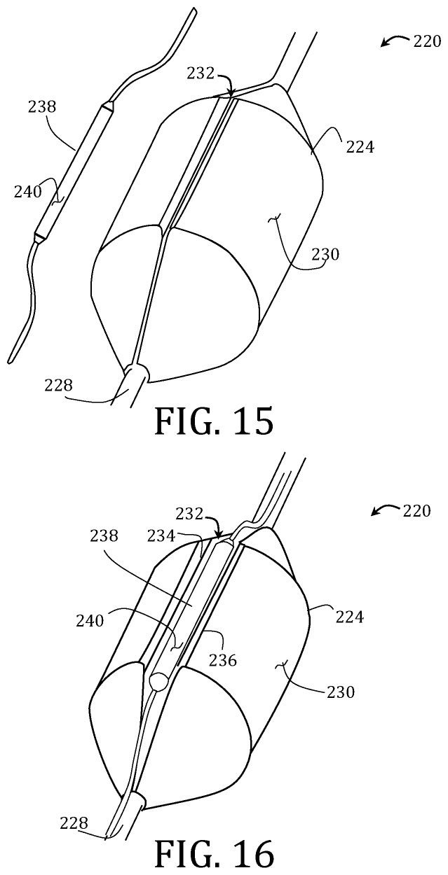

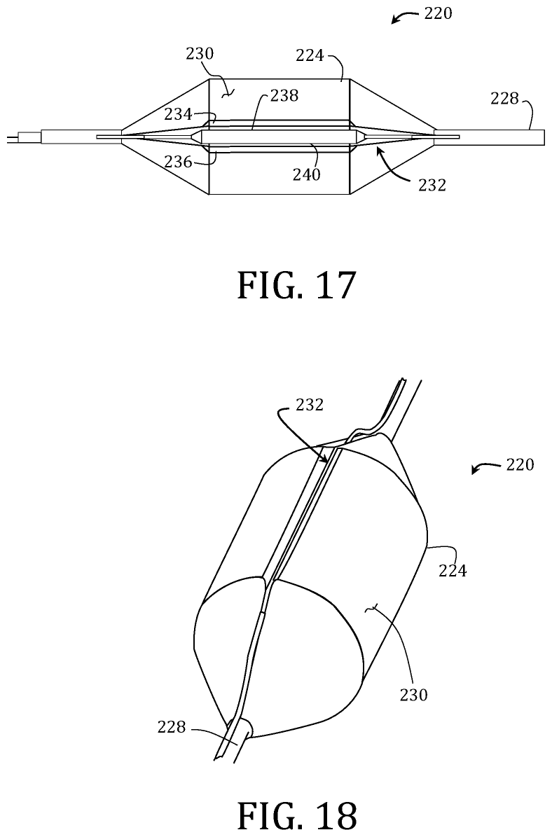

Turning now to FIGS. 14-18, an occluding portion 220 of an endovascular occlusion device 222 according to another embodiment of the present invention is described. The occluding portion 220 includes a compressible, occluding balloon 224 on a distal end 226 of a catheter 228 having a lumen (not shown). The lumen may include a multiple passages therein, one of such passages may be configured to receive and be in sliding relation to the guidewire 110. Another of such passages may be configured to receive an inflation fluid and is in fluid communication with the occluding balloon 224. The occluding balloon 224, therefore, is configured such that an outer surface 230 thereof, after inflation, may contact the lumen of the vessel in which the occlusion portion is positioned.

The occluding balloon 224 includes a channel 232 extending a portion of the length thereof and radially inwardly from the outer surface 230 toward the catheter 228. Sides 234, 236 of the channel 232 may include, be constructed of, or incorporate a non-compliant material configured to provide a degree of rigidity to the channel 232.

A non-compliant balloon 238 is positioned within the channel of the occluding balloon 224. A length of the non-compliant balloon 238 may, although not required, be substantially similar to a length of the channel 232 and is configured such that an outer surface 240, with inflation, moves from a minimum diameter to a diameter sufficient to force the sides 234, 236 of the channel 232 to move in opposing directions such that the non-compliant balloon 238 operates as a wedge within the channel 232.

While not required, and not explicitly illustrated herein, the non-compliant balloon 238 may be coupled to the occluding balloon 224 such that the non-compliant balloon 238 and the occluding balloon 224 are more easily movable as a singular unit.

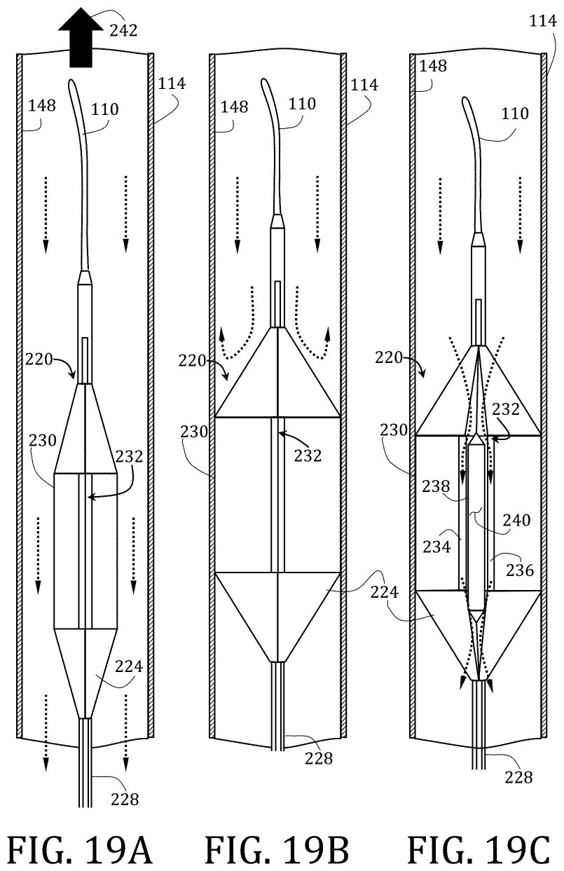

In use, as shown in FIGS. 19A-19C with reference to FIG. 1, the guidewire 110 may be inserted into a primary incision site 102 and navigated through the vasculature to an occlusion site. With the guidewire 110 in place, the occluding portion 220 of the occlusion device 222, while deflated, may be advanced over the guidewire 110 (in a direction of the arrow 242) to the occlusion site (FIG. 19A). When suitable or appropriately positioned at the occlusion site, the occluding balloon 224 may be inflated such that an outer surface 230 of the occluding balloon 224 contacts the inner wall 148 of the abdominal aorta 114, thereby securing the occluding portion 220 within the lumen 154 of the abdominal aorta 114. As explicitly illustrated in FIG. 19B, blood flow through the abdominal aorta 114 is stopped with the fully inflated occluding balloon 224 contacting the inner wall 148 (see dashed arrows).

When blood flow is desired or necessary, as illustrated in FIG. 19C, the non-compliant balloon 238 may be inflated such that the outer surface 240 contacts the sides 234, 236 of the channel 232 of the occluding balloon 224, thereby opening the channel 232 to a degree related to a degree of inflation of the non-compliant balloon 238.

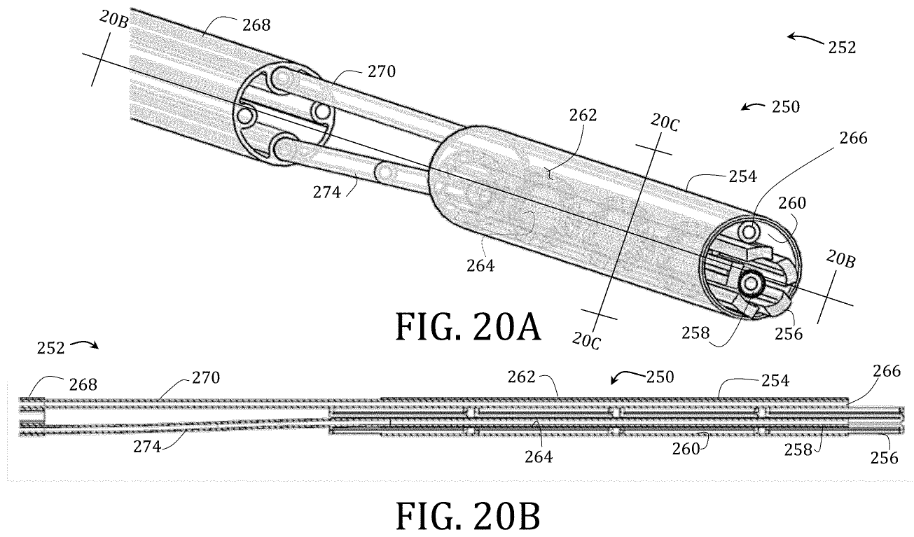

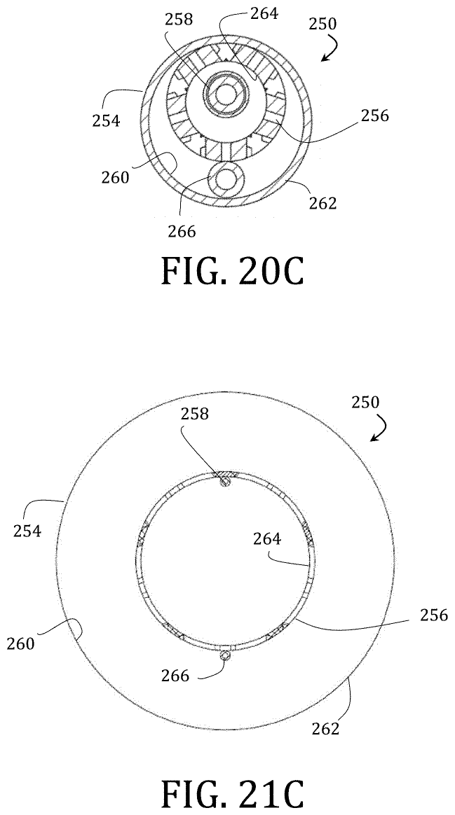

Turning now to FIGS. 20A-23B, an occluding portion 250 of an endovascular occlusion device 252 according to still another embodiment of the present invention is shown. The occluding portion 250 includes a first balloon 254, a stent 256, and second balloon 258 arranged coextensively (and in some other embodiments, collinearly, coaxially, or both). More particularly, the stent 256, which may be custom fabricated by laser cutting stainless steel or Nitinol or any commercially-available, self-expanding, covered, endovascular stent graft, such as the FLAIR manufactured by Bard Peripheral Vascular (Tempe, Ariz.) or the covered WALLSTENT by Boston Scientific (Natick, Mass.), is positioned with a lumen 260 of the first balloon 254. The first balloon 254 may be constructed from a compliant or non-compliant material, and an outer surface 262 thereof is configured to, when inflated, contact the inner wall of the vessel in which the occluding portion 250 is positioned.

The second balloon 258 is positioned within a lumen 264 of the stent 256 and may be constructed from a non-compliant material so as to facilitate deploying of the stent 256 within the lumen 260 of the first balloon 254.

A removably coupled shaft 266, as specifically shown in FIGS. 20A-20C, extends into the lumen 260 of the first balloon 254 and is configured to receive and move in sliding relation to the guidewire 110 (FIG. 1). While not specifically illustrated herein, a lumen of the second balloon 258 may be constructed to receive and move in sliding relation to the guidewire 110 (FIG. 1), similar to previously described embodiments.

A catheter hub 268 extends proximally away from the occluding portion 250 and is configured to support an inflation line 270 for the first balloon 254, control wires 272 operably coupled to the stent 256, an inflation line 276 for the second balloon 258, and the shaft 266 for receiving the guidewire 110 (FIG. 1).

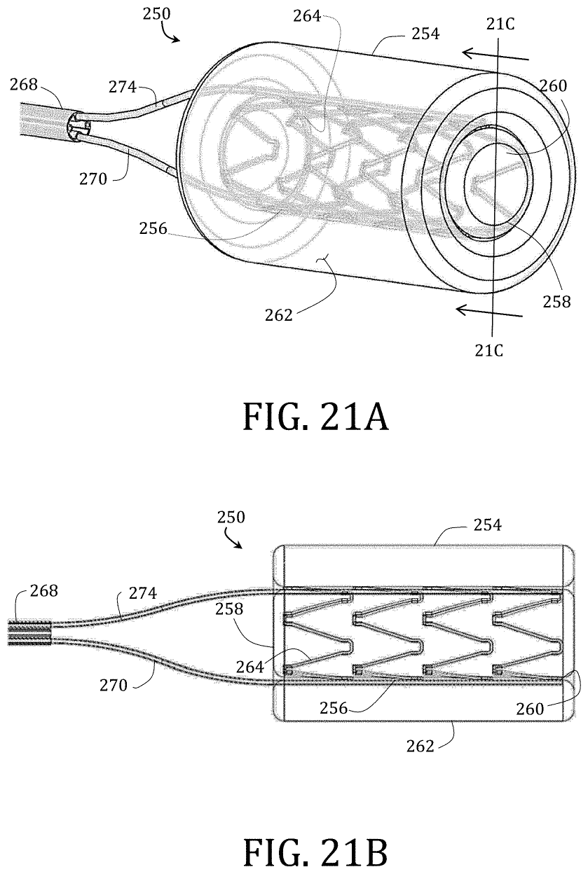

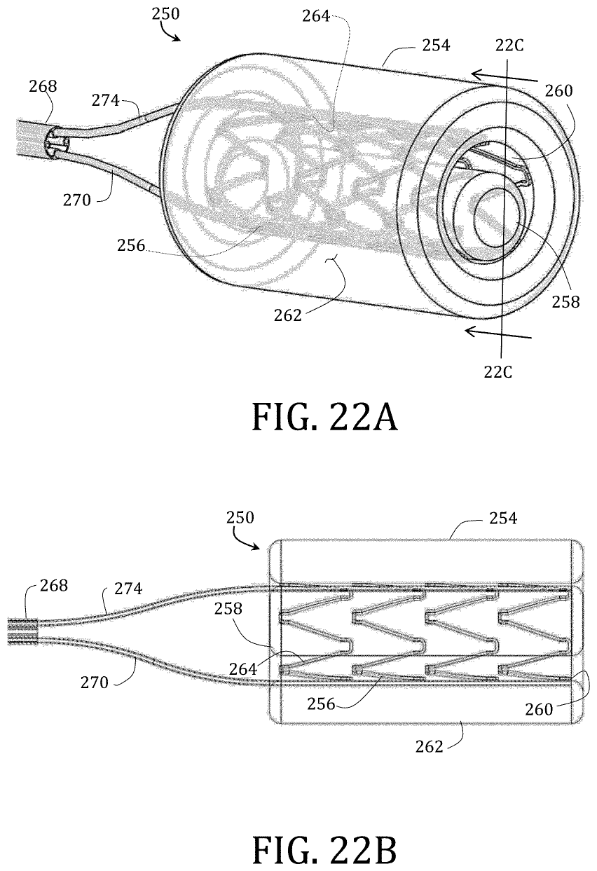

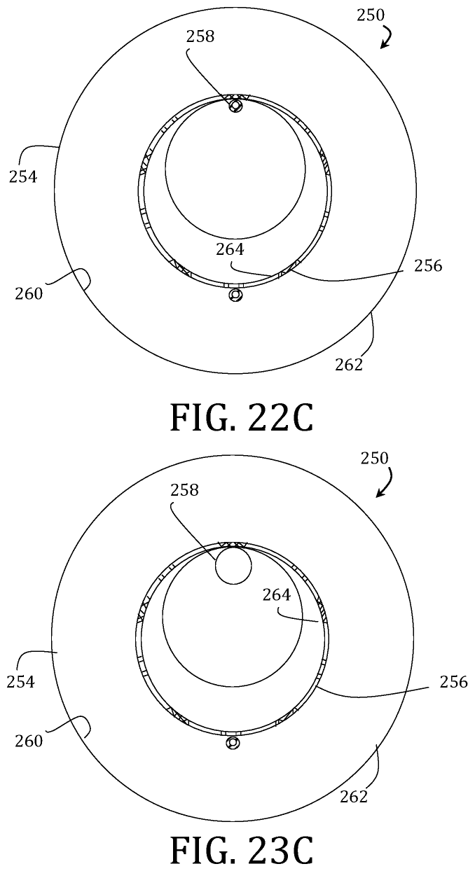

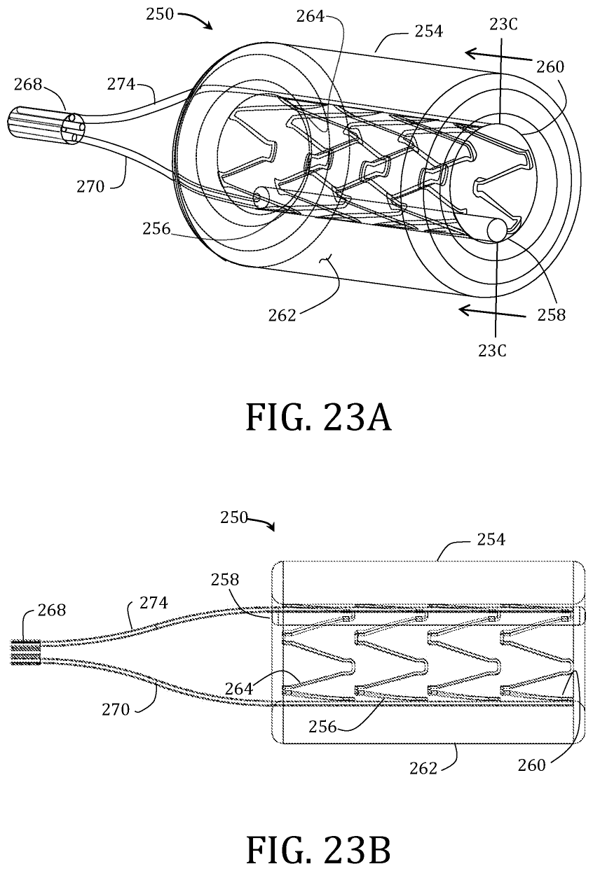

Referring to FIGS. 21A-21C, the shaft 266 for the guidewire 110 (FIG. 1) has been removed and the first balloon 254, the stent 256, and the second balloon 258 are inflated (or deployed as with respect to the stent 256) each to its maximum diameter. In FIGS. 22A-23C, while a diameter of the second balloon 258 decreases with deflation, the stent 256 remains deployed so as to support the shape and position of the first balloon 254 within the vasculature.

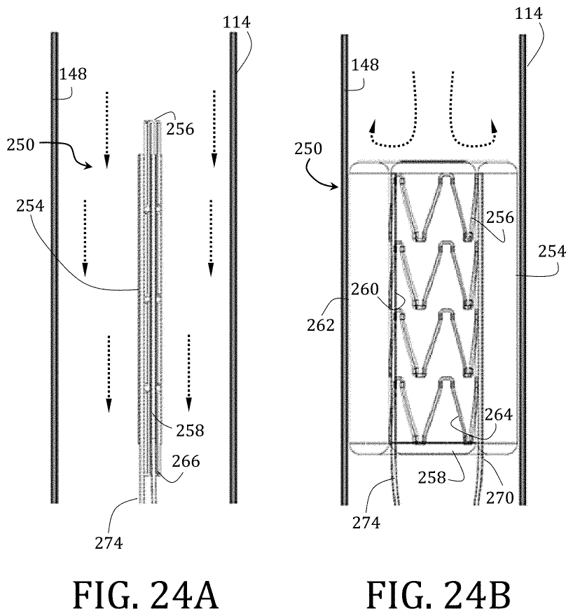

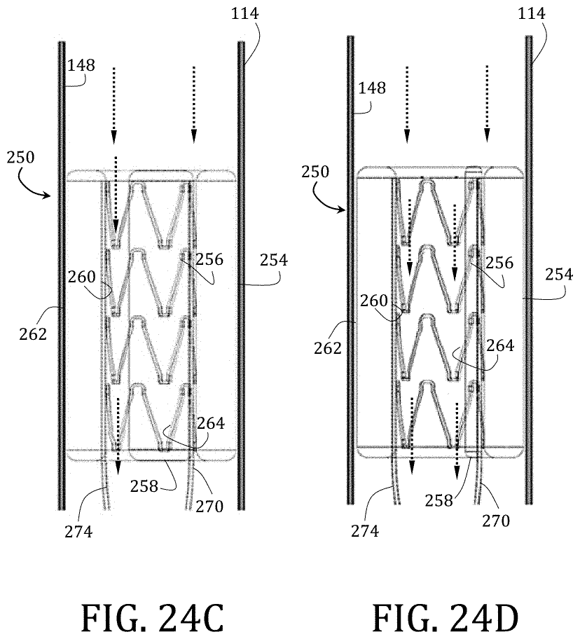

Referring now to FIGS. 24A-24D with reference to FIG. 1, a method of using the occluding portion 250 illustrated in FIG. 20A according to an embodiment of the present invention is shown. At start, and as described above, the guidewire 110 may be inserted into a primary incision site 102 and navigated through the vasculature to an occlusion site.

With the guidewire 110 in place, the endovascular occlusion device 252 may be back-loaded and advanced over the guidewire 110 to the occlusion site. As shown in FIG. 24A, the occluding portion 250 of the occlusion device 252 is positioned at the occlusion site and the guidewire 110 retracted.

In FIG. 24B, when the occluding portion 250 is suitable or appropriately positioned at the occlusion site, the first balloon 254 may be inflated such that an outer surface 262 of the first balloon 254 contacts the inner wall 148 of the abdominal aorta 114, thereby securing the occluding portion 250 within the lumen 154 of the abdominal aorta 114. The second balloon 258 is also inflated such that the stent 256 is fully deployed within the lumen 260 of the first balloon 254.

FIGS. 24B-24D illustrate varying degrees of occlusion, wherein FIG. 24B illustrates full occlusion, and 24D illustrates minimal occlusion achievable without removing the occluding portion 250. In this way, a degree of blood flow (illustrated with dashed lines) may be achieved and is related to a degree of inflation of the second balloon 258.

When the endovascular occlusion device of FIGS. 24A-24D is to be withdrawn and retracted from the occluding site, retraction on the control wires 272 of the stent 256 cause retraction and collapse of the stent 256. With the stent 256 withdrawn, the first balloon 254 may be deflated and likewise retracted.

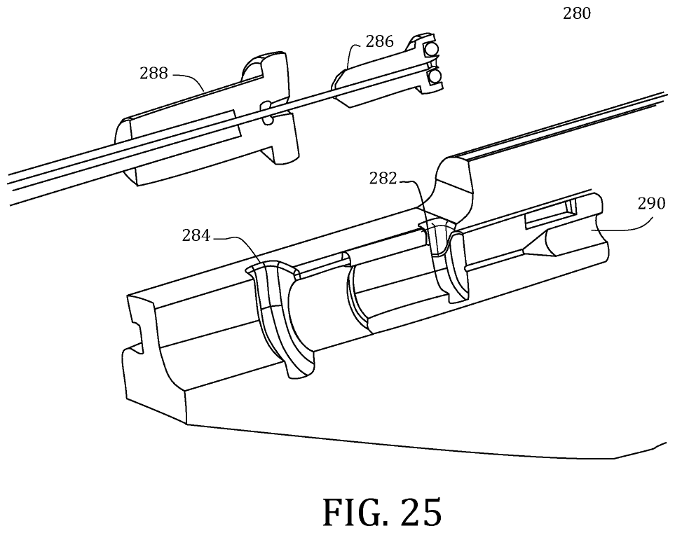

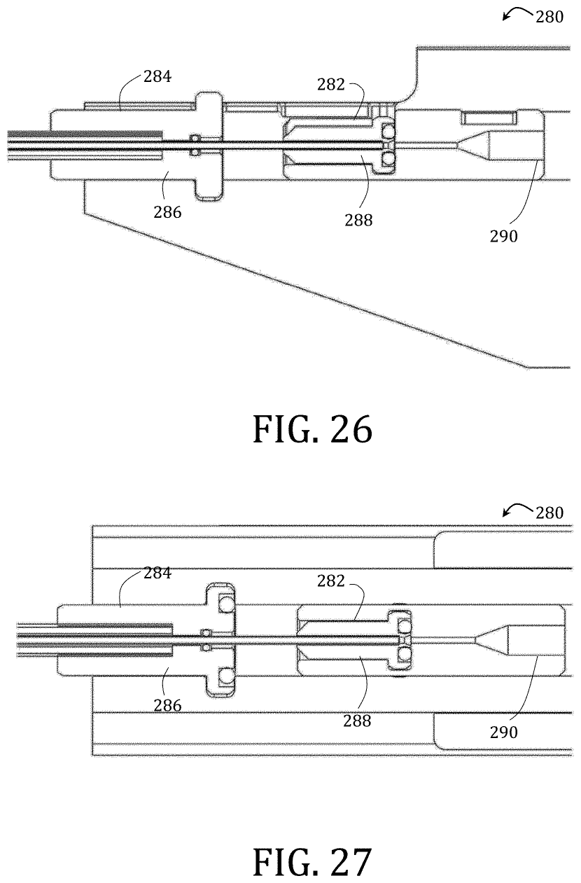

Because of the number of catheters 254, 258, stents 256, control wires 272, and shafts 266 associated with the endovascular occlusion device 252 of FIG. 20A, it is necessary to maintain control and separate manipulation of each element. FIGS. 25-27 illustrates one such suitable control handle 280 according to an embodiment of the present invention. The control handle 280 includes a first port 282 and a second port 284 configured to receive first and second hubs 286, 288 operably coupled to one or more of the inflation lines 270, 274, the control wires 272, the shaft 266, or other auxiliary devices as would be used by the skilled surgeon.

As illustrated, the hubs 286, 288 may be arranged in series to minimize an overall diameter of the control handle. More particularly, a primary port 290 may be centrally disposed and is configured to provide a primary supply of inflation fluids, for example.

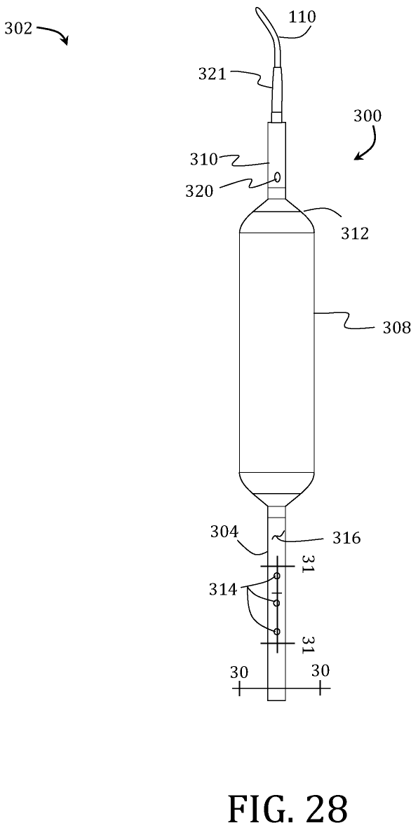

Turning now to FIGS. 28-31, an occluding portion 300 of an endovascular occlusion device 302 according yet another embodiment of the present invention is shown and includes a flow port catheter 304 having a balloon 306 coupled to a distal end 308 thereof. A distal tip 310 of the flow port catheter 304 extends beyond a distal end 312 of the balloon 306.

The flow port catheter 304, proximal to the balloon 306, includes a plurality of ports 314 extending from a surface 316 to a lumen 318 of the catheter 304 to provide fluid communication therebetween. In a similar manner, the distal tip 310 of the flow port catheter 304 may include at least one port 320 that also extends from the surface 312 to the lumen 318 of the catheter 304.

While shown in FIG. 28, although not required, an obturator 321 may be used for introducing or advancing the occluding portion 300 as is known in the art.

The balloon 306 may be constructed for a compliant or semi-compliant material and is configured to move from a deflated state to an inflated state. When in the inflated state, an outer surface 322 of the balloon 306 may contact an inner wall of the vascular in which it is positioned.

A flow restrictor 324 is disposed within the lumen 318 of the flow port catheter 304 and is in sliding relation thereto. The flow restrictor 324 may be constructed from a non-compliant material and has a length that is sufficient to extend over all ports 314 proximal to the balloon 306 but is also sufficiently shortened such that the flow restrictor 324 may be advance distally within the lumen of the flow port catheter 304 to expose one or more of the ports 314.

The flow restrictor 324 may include a lumen 326 configured to receive and move in sliding relation to a guidewire 110 (FIG. 1), in a manner similar to what was described previously. Moreover, as the flow restrictor 324 is shortened and thus does not extend the length of the catheter 304 to the primary incision site 102 (FIG. 1), one or more control wires 328 may extend proximally from a distal end of the flow restrictor 324 to the handle 120 (FIG. 1) for manipulation thereof.

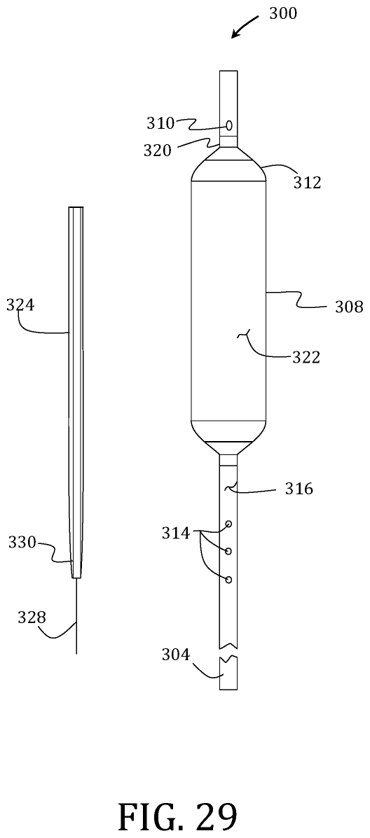

In the particular illustrative embodiment of FIG. 29, a proximal end of the flow restrictor 324 may include a tapered surface 330; however, such shape is not required.

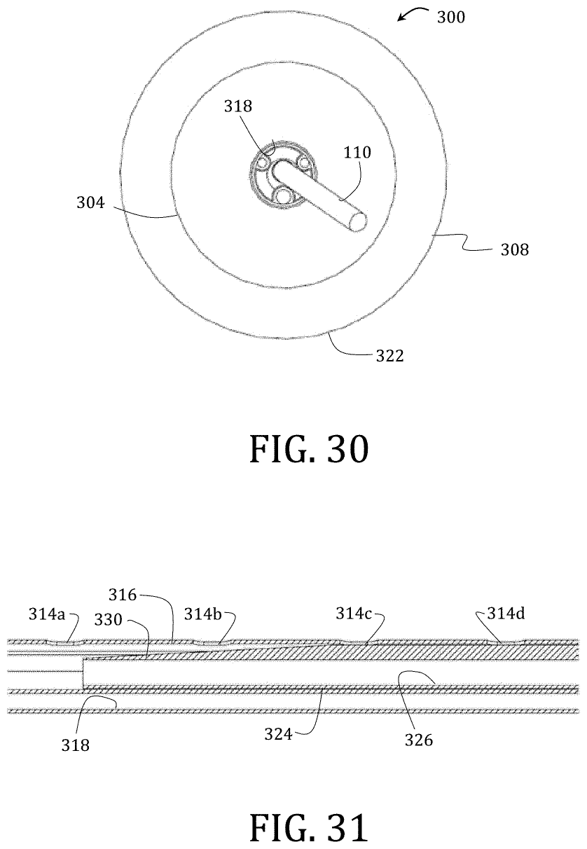

FIG. 30 is a cross-sectional view of the flow restrictor 324 taken along the line 30-30 in FIG. 28. As shown, the guidewire 110 extends through a central lumen. Additional lumens are provided for inflation fluid, sensors, and so forth.

FIG. 31 is a cross-section view of the flow port catheter 304 and the flow restrictor 324 taken along the line 31-31 of FIG. 28. Four ports 314a, 314b, 314c, 314d of the flow port catheter 304 are shown. The tapered surface 330 of the flow restrictor 324 is positioned proximate to the first port 314a; however, the first port 314a is open so as to permit fluid flow between the surface 316 of the catheter 304 and the lumen 318 of the catheter 304. The remaining ports 314b, 314c, 314d are, in effect, closed as the flow restrictor is adjacent thereto. Movement of the flow restrictor 324 in a direction (arrow 332) causes the tapered surface 300 to move past the second port 314b, the third port 314c, and so forth. Such movement, therefore, increases a level of flow between the surface 316 and the lumen 318 of the catheter 304.

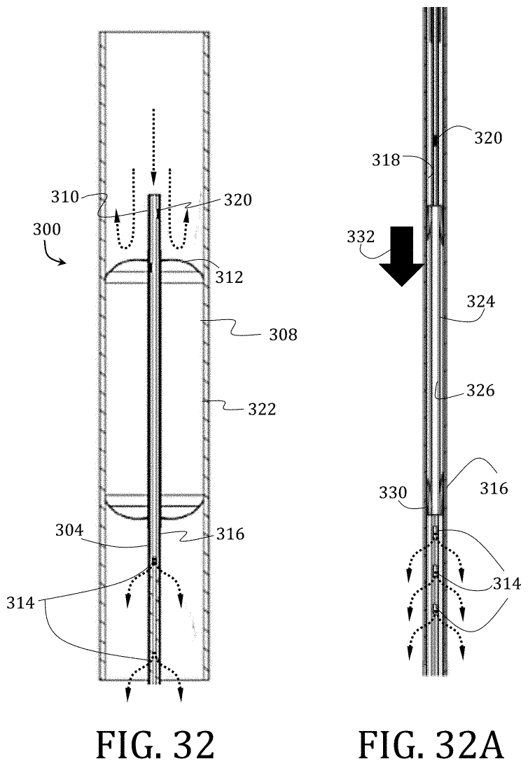

Referring now to FIGS. 32-35A with reference to FIG. 1, methods of using the occluding portion 300 illustrated in FIG. 28 according to embodiments of the present invention are shown. At start, and as described above, the guidewire 110 may be inserted into a primary incision site 102 and navigated through the vasculature to an occlusion site.

With the guidewire 110 in place, the endovascular occlusion device 302 may be back-loaded and advanced over the guidewire 110 to the occlusion site. Once suitably positioned, the balloon may be inflated such that the outer surface 322 of the balloon 306 contacts the inner wall 148 of the abdominal aorta 114, thereby securing the occluding portion 300 within the lumen 154 of the abdominal aorta 114.

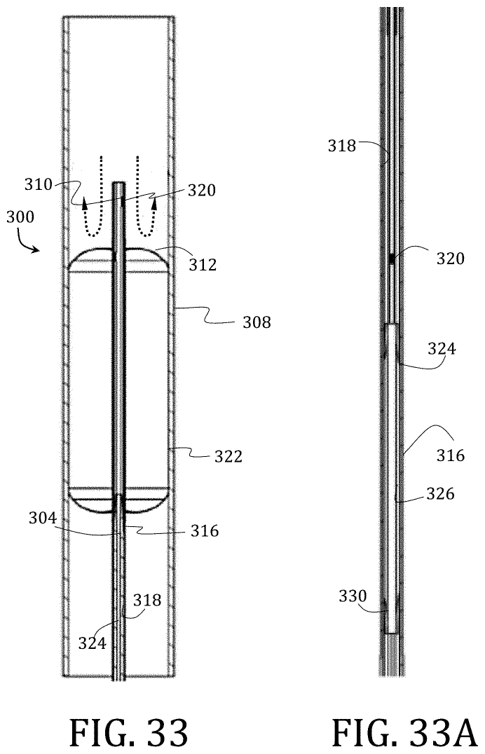

Use of the occluding portion 300 illustrated in FIG. 28 in retrograde blood flow is described with reference to FIGS. 32 and 33. In FIG. 32, blood flow enters the distal tip 308 of the flow port catheter 304 and exits the lumen 318 of the catheter 304 at the open ports 314. FIG. 32A is a cross-sectional view of a positioning of the flow restrictor 324 relative to the flow port catheter 304, as illustrated in FIG. 32.

Retracting the flow restrictor 324 within the lumen 318 of the flow port catheter 304, as shown in FIG. 33A, causes the flow restrictor 324 to cover the ports 314. As such, blood flow through the lumen 318 of the flow port catheter 304 is restricted.

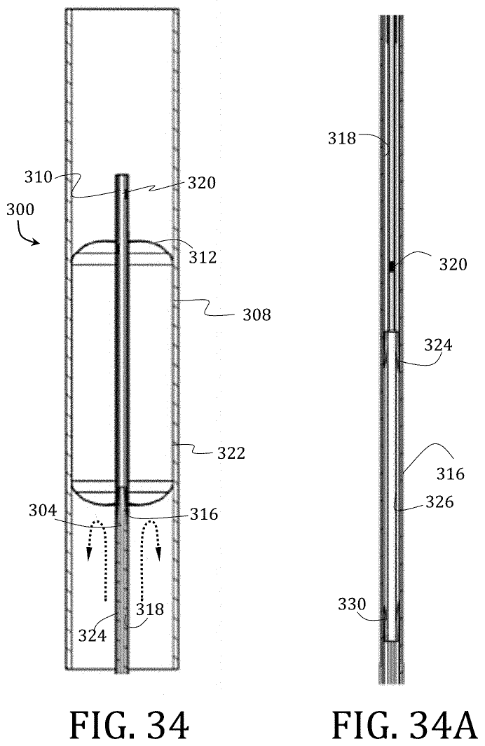

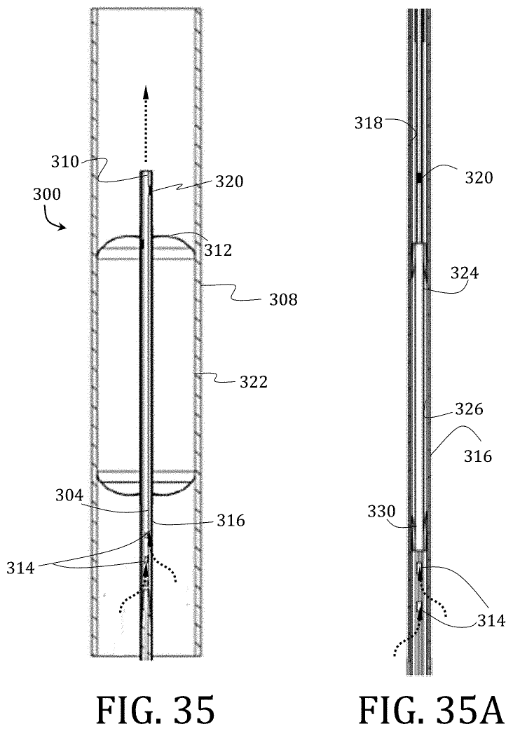

Use of occluding portion 300 illustrated in FIG. 28 in anterograde blood flow is described with reference to FIGS. 34 and 35. In FIG. 34, the flow restrictor 324 within the lumen 318 of the flow port catheter 304, as shown in FIG. 34A, causes the flow restrictor 324 to cover the ports 314. As such, blood flow through the lumen 318 of the flow port catheter 304 is restricted.

When the flow restrictor 324 is advanced within the lumen of the flow port catheter 304, as shown in FIG. 35A, blood flow enters the open ports 314 of the flow port catheter 304 and exits the lumen 318 of the catheter at the distal tip 308.

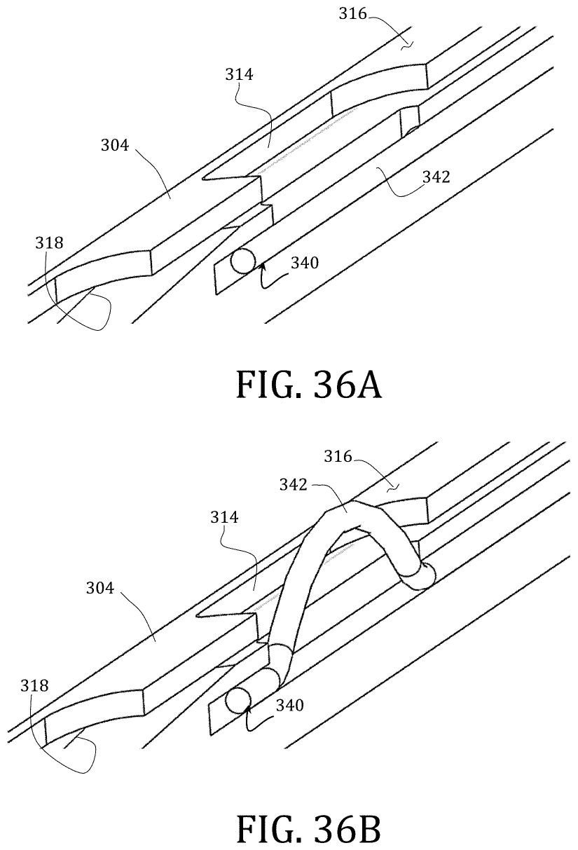

During use of the occluding portion 300 illustrated in FIG. 28, it may become necessary to clear one or more ports 314, 320. Clogging of the ports 314, 320 may occur by clotting of blood if blood flow remains stagnant within the lumen 318 of the catheter 304 for a period of time. A method of clearing the ports 314, 320 according to one embodiment of the present invention is shown in FIGS. 36A and 36B. In that regard, a flexible appliance, for example constructed from a memory-shape metal or a shape-memory polymer, may be advanced through the lumen 318 of the flow port catheter 304 to a clogged port. Because the appliance is made of shape-memory materials, a laterally-deflecting portion of the appliance automatically springs radially outwardly through the port 314, 320. Collapsing the laterally-deflecting portion may occur by advancing or retracting the appliance beyond the port 314, 320.

While the laterally-deflected portion is shown to have a semi-circular shape, it would be readily understood by those having ordinary skill in the art and the benefit of the disclosure made herein that such illustrative shape need not be limiting.

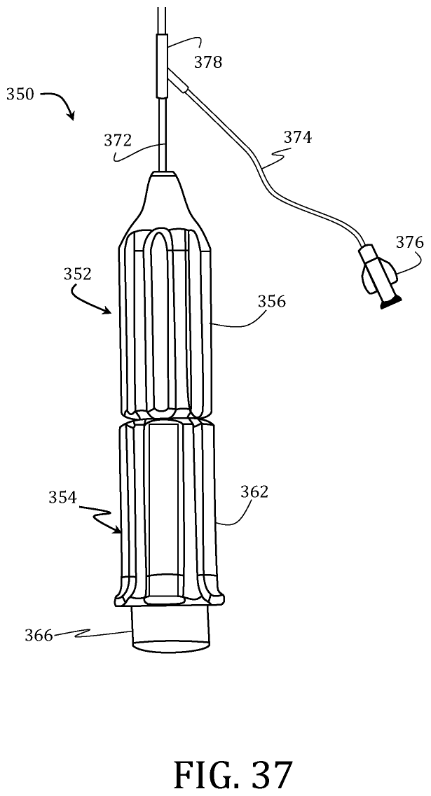

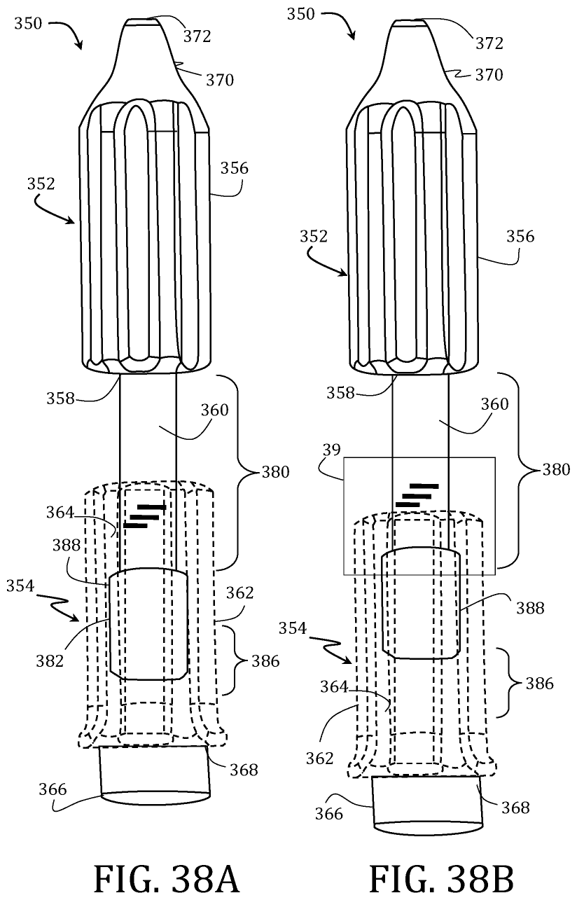

Referring now to FIGS. 37-38B a control handle 350 suitable for use with the occluding portion 300 of FIG. 28, according with an embodiment of the present invention, is shown, and includes a distal handle 352 and proximal handle 354. The distal handle 352 includes a grip collar 356 coupled to a distal end 358 of a shaft 360. The proximal handle 354 includes a grip collar 362 and a lumen 364 configured to receive the shaft 360 of the distal handle 352. A proximal hub 366 is coupled to a proximal end 368 of the proximal handle 354 and is configured to receive one or more catheters, lumen, guidewires, and other like instruments conventionally used in endovascular surgeries.

A proximal tip 370 of the distal handle 352 is configured to receive a shaft 372, catheter, sheath, or other like device that is operably coupled to one of more surgical devices. For purposes of illustration herein, the surgical device is the occluding portion 300 of FIG. 28. As a result, the shaft 372 may include multiple lumen or channels for managing the surgical devices. One such lumen may provide passage of an inflation line (not shown) of the balloon 306 (FIG. 28). An externally positioned inflation line 374 with luer lock 376 may be coupled to the inflation line lumen of the shaft 372 by way of a y-joint 378, all configured to provide fluid communication with the balloon 306 (FIG. 28). Another such lumen may provide passage of the control wire 328 (FIG. 29) operably coupled to the flow restrictor 324 (FIG. 29) within the flow port catheter 304 (FIG. 28). Still other such lumen may be used for housing sensors or other like surgical instruments.

As illustrated in FIGS. 38A and 38B, the shaft 360 of the distal handle 352 includes a graduated slide 380 and a threaded cap 382 on a proximal end 384 of the graduated slide 380. Likewise, the lumen 364 of the proximal handle 354 includes a smooth portion 386 and a threaded lumen 388 that is distal to the smooth portion 386 and configured to receive the threaded cap 382 of the distal handle 352.

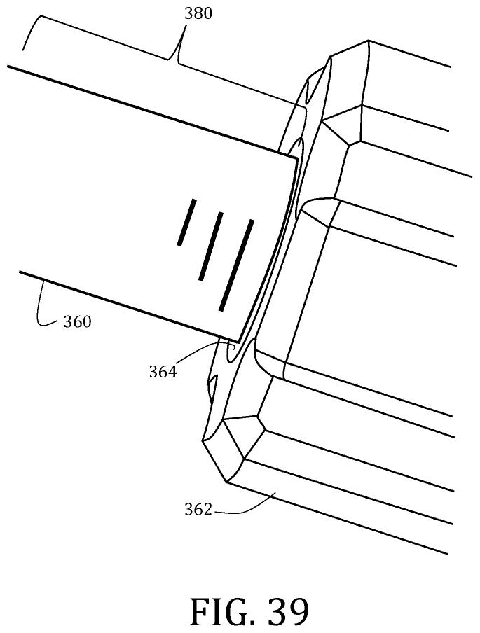

The graduated slide 380 may include indicia (illustrated as lines, with an enlarged view provided in FIG. 39) of measurements that may reflect a linear translation of an associated surgical device. In that regard, use of the control handle 350 may proceed by advancing the distal handle 352 distally from the proximal handle 354 such that the graduated slide 380 moves in sliding relation to, and out from within, the smooth portion 386 of the proximal handle 354 until the threaded cap 382 of the distal handle 352 contacts the threaded lumen 388 of the proximal handle 354. When this contact between threaded cap 382 and threaded lumen 388 is made, the indicia of the graduated slide 380 may be visible between the grip collars 356, 362 of the distal and proximal handles 352, 354. Such sliding movement may be used to advance the flow restrictor 324 (FIG. 29) into the flow port catheter 304 (FIG. 28) near the ports 314 (FIG. 28).

Further advancing of the flow restrictor 324 (FIG. 29) with respect to the flow port catheter 304 (FIG. 28) may be accomplished by rotating the distal handle 352 with respect to the proximal handle 354 (or vice versa). The rotational movement causes a linear advancing or retracting (depending on whether direction or rotation and direction of threading) of the flow restrictor 324 (FIG. 29).

According to some embodiments, the indicia of the graduated slide 380 may indicate a distance advanced or retracted by the flow restrictor 324 (FIG. 29). In other embodiments, the indicia may reflect positioning of the flow restrictor 324 (FIG. 29) with respect to the ports 314 (FIG. 28) of the flow port catheter 304 (FIG. 28).

While not specifically illustrated, one of ordinary skill in the art would understand that the threaded cap 382 and the threaded lumen 388 may be replaced with other known mechanical systems suitable for adjusting linear displacement. A suitable alternative may be, for example, a ratchet.

While not explicitly illustrated herein, one of more of the embodiments of the present invention described herein may incorporate additional tools that are conventionally used in endovascular procedures. For example, a delivery sheath may be use to enclose the endovascular occlusion device so as to facilitate delivery of the device to the occluding site. Such suitable delivery sheaths may include a 7-9 French sheath. Moreover, the guidewires may include any suitable or preferred guidewire type, whether a j-loop, coil, and so forth.

One or more pressure sensors may be used with endovascular occlusion devices according to any embodiment of the present invention described herein. The pressure sensors may be configured to communicate blood pressure, measured locally, to an external display. Such blood pressure information may assist the surgeon in making operational decisions. Additionally or alternatively, the blood pressure information may be processed by an external control devices so as to adjust flow restriction. For example, a rotary or stepper motor operably coupled to such external control devices may be operable to inflate/deflate balloons, reposition flow restrictors, advance/retract delivery sheaths, and so forth. The external control devices may also incorporate an algorithm configured to determine a physiological status of the patient given the blood pressure information with or without additional measurements.

While embodiments of the present invention were envisioned as fulfilling a need associated with the treatment of soldiers injured in the battlefield, embodiments of the present invention have applicability beyond the battlefield. Any patient having a significant risk of hemorrhage may benefit from use of an endovascular occlusion device as described according to various embodiments herein.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Example

A prototypical endovascular occlusion device similar to the embodiment illustrated in FIG. 28 was evaluated for flow rate and pressure. In that regard, a syringe with pressure gauge were coupled to the proximal end of the balloon catheter. Three ports were included in the flow port catheter.

Backpressure was evaluated using a pig model comprising a 12.7 mm ID×1.5 mm wall silicone tubing (aorta), a flow regulator downstream of the “aorta,” and two pressure gauges on opposing ends of the aorta. Table 1 summarizes measured flow measurements and backpressures:

TABLE 1

| | Flow Measurements | | | |
|---|---|---|---|---|
| | | Flow Rate | Flow Rate | |
| | | 0 mm Hg distal | 40 mm Hg distal | Delta |
| | # Holes | (mL/min) | (mL/min) | (%) |
| ΔP = | 1 | 138 | 133 | −3.6 |
| 75 mm Hg | 2 | 215 | 223 | 3.9 |
| | 3 | 317 | 302 | −4.7 |
| | 4 | 398 | 390 | −2.1 |
| | 5 | 415 | 423 | 2.0 |
| | 6 | 455 | 448 | −1.5 |
| ΔP = | 1 | 188 | 180 | −4.4 |
| 130 mm Hg | 2 | 300 | 298 | −0.6 |
| | 3 | 442 | 420 | −4.9 |
| | 4 | 527 | 553 | 5.1 |
| | 5 | 575 | 572 | −0.6 |
| | 6 | 605 | 610 | 0.8 |

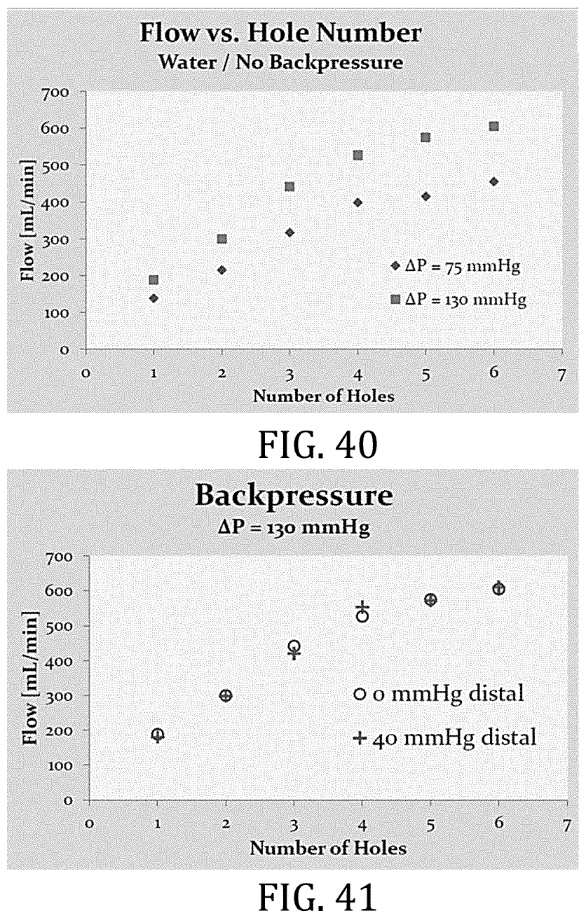

Data of Table 1 are graphically illustrated in FIGS. 40 and 41. From Table 1, it was concluded that change in pressure drives flow and backpressure was negligible.

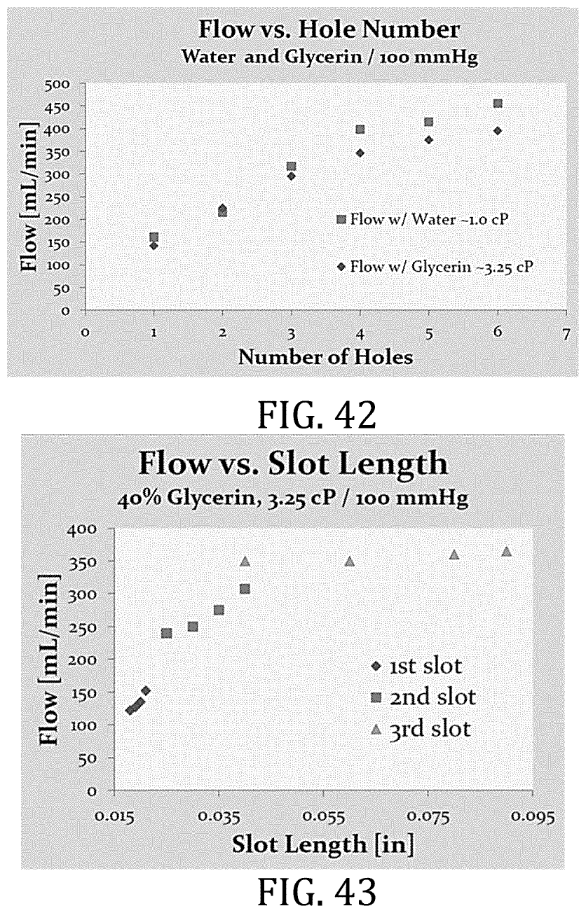

The experiments were repeated with 40% glycerin and compared with the results for water. Table 2, below, summarizes the data. Data is also illustrated graphically in FIGS. 42 and 43.

TABLE 2

| | Flow with water | Flow with glycerin | |
|---|---|---|---|
| | ~1.0 cP | ~3.25 cP | Delta |
| Hole | (mL/min) | (mL/min) | (%) |
| 1 | 161 | 142 | 11.8 |
| 2 | 215 | 225 | −4.7 |
| 3 | 317 | 295 | 6.8 |
| 4 | 398 | 346 | 13.1 |
| 5 | 415 | 375 | 9.6 |
| 6 | 455 | 395 | 13.2 |

ΔP = 100 mm Hg

As described herein, embodiments of the present invention provide endovascular occlusion while maintaining the ability to allow for controlled distal (anterograde) blood flow to varying degrees. The endovascular device described herein is configured to allow anterograde blood flow rates ranging from about 5% to about 10% of baseline blood flow, which ameliorate the deleterious effects of prolonged distal ischemia.

Endovascular occlusion devices configured to permit anterograde blood flow rates ranging from 5% to 10% of baseline blood flow are describe herein according to embodiments of the present invention. Permitting such anterograde flow during conventional endovascular occlusion procedures have been shown to ameliorate deleterious effects of prolonged distal ischemia. Such devices may provide minimally invasive procedures for treating non-compressible torso hemorrhage and shock.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. An endovascular occlusion device comprising:
a first balloon having a distal end, a proximal end, and a lumen extending therebetween, the first balloon having a deflated state and an inflated state, wherein the first balloon in the inflated state is configured to contact an inner wall of a vasculature;
a second balloon having a distal end, a proximal end, and a lumen extending therebetween, the second balloon being coaxial with the first balloon and having a deflated state and an inflated state; and
an inflatable plug having a distal end and a proximal end, the inflatable plug being coaxial with the first and second balloons and having a deflated state and an inflated state,
wherein the inflatable plug, while in the inflated state, is configured to form a seal with the second balloon.

2. The endovascular occlusion device of claim 1, wherein the first balloon is constructed from a compliant material or a non-compliant material and the second balloon and the inflatable plug are constructed from a non-compliant material.

3. The endovascular occlusion device of claim 1, wherein the distal end of the inflatable plug forms the seal with the proximal end of the second balloon.

4. The endovascular occlusion device of claim 1, wherein the proximal end of the inflatable plug forms the seal with the distal end of the second balloon.

5. The endovascular occlusion device of claim 1, further comprising:
a handle operable coupled to the first and second balloons and the inflatable plug.

6. The endovascular occlusion device of claim 3, further comprising:
a sheath having a proximal end, a distal end, and a lumen extending therebetween, the proximal end of the sheath being operably coupled to the handle and the distal end of the sheath being operably coupled to the first and second balloons and the inflatable plug.

7. The endovascular occlusion device of claim 6, wherein the sheath includes a plurality of lumens.

8. The endovascular occlusion device of claim 1, further comprising:
a guide wire configured to extend through a lumen of the first balloon.

9. The endovascular occlusion device of claim 1, further comprising:
a delivery sheath configured to surround and receive the first and second balloons and the inflatable plug.

10. A method of using the endovascular occlusion device of claim 1, the method comprising:
positioning the endovascular occlusion device in a blood vessel having anterograde blood flow, wherein the endovascular occlusion device is configured to restrict a rate of blood flow through the blood vessel to 5% to 10% of a baseline rate.

11. A method of using the endovascular occlusion device of claim 1, the method comprising:
positioning the endovascular occlusion device in a blood vessel having anterograde blood flow, wherein the endovascular occlusion device is configured vary a rate of blood flow through the blood vessel from no blood flow, up to 10% of a baseline rate, or the baseline rate.
